(19) United States
(12) Patent Application Publication
(10) Pub. No.: US 2004/0002647 A1
(43) Pub. Date: Jan. 1, 2004

(54) Gel injection treatment of body parts

(76) Inventor: Ashvin Desai, San Jose, CA (US)

Correspondence Address: David H. Jaffer, Pillsbury Winthrop LLP, 2550 Hanover Street, Palo Alto, CA, 94304-1115, US

(21) Appl. No.: 10/193,721

(22) Filed: Jul. 10, 2002

Related U.S. Application Data
(63) Continuation-in-part of application No. 09/715,853, filed on Nov. 17, 2000.
(63) Continuation-in-part of application No. 09/510,537, filed on Feb. 22, 2000, now Pat. No. 6,461,296.
(63) Continuation-in-part of application No. 09/105,896, filed on Jun. 26, 1998, now Pat. No. 6,231,591.
(63) Continuation-in-part of application No. 08/639,199, filed on Apr. 26, 1996, now Pat. No. 5,861,002.
(63) Continuation-in-part of application No. 08/259,712, filed on Jun. 14, 1994, now Pat. No. 5,562,703.
(63) Continuation-in-part of application No. 08/025,003, filed on Mar. 2, 1993, now abandoned.
(63) Continuation-in-part of application No. 07/779,108, filed on Oct. 18, 1991, now Pat. No. 5,322,503.
(60) Provisional application No. 60/383,015, filed on May 23, 2002.

(51) Int. Cl. A61B 5/05

(52) U.S. Cl. 600/417

(57) ABSTRACT

A method and apparatus for treating disease by injecting a treatment substance directly into the diseased body part, and thereby leaving the remaining body parts relatively unaffected. Specific treatment substance formulations are provided for each of a plurality of body parts for specific diseases. The treatment substance formulations contain two principle parts including a preferred active treatment (therapy) substance, and a preferred inactive binding (carrier) substance for thickening the treatment substance such as a specific gel or viscous material for carrying the preferred active treatment (therapy) substance for a particular disease and body part. The method also provides preferred treatment substance dosages to be injected into each body part. Apparatus for injecting the treatment substance is also provided that can be used with endoscopic instruments.

### RELATED CASES

The application claims priority from U.S. Provisional Patent Application No. 60/383,015, filed May 23, 2002, and is a continuation-in-part of U.S. patent application Ser. No. 09/715,853 filed Nov. 17, 2000 which is a continuation-in-part of U.S. patent application U.S. patent application Ser. No. 09/510,537 filed Feb. 22, 2000, which is a continuation-in-part of U.S. patent application Ser. No. 09/105,896 filed Jun. 26, 1998, which is a continuation-in-part of U.S. patent application Ser. No. 08/639,199 filed Apr. 26, 1996, which is a continuation-in-part of U.S. patent application Ser. No. 08/259,712 filed Jun. 14, 1994, which is a continuation-in-part of U.S. patent application Ser. No. 08/025,003 filed Mar. 2, 1993; which is a continuation-in-part of U.S. patent application Ser. No. 07/779,108 filed Oct. 18, 1991. The contents of each of these applications is incorporated in this application by reference.

### BACKGROUND OF THE INVENTION

1. Field of the Invention

The present invention relates generally to methods and apparatus for treating diseases of body parts, and more specifically to treatment involving the injection of a substance of a particular composition and formulation into a body part for treatment of a specific disease condition.

2. Description of the Prior Art

A variety of treatment fluids are currently known to be of benefit in treating illness in particular body parts. For example, there are a number of tumor suppressor genes, viral vectors, markers, vaccines, enzymes, proteins and biological agents that can be used for gene therapy and cancer treatment. The traditional method of delivery of these substances is to inject them into the blood steam through use of a conventional needle and syringe. This approach severely limits the formulation and concentration of substances that can be applied for treatment of a particular body part, because the entire body is subjected to the substance, and therefore must be able to tolerate the application. Because of this, the existing formulations for treatment are confined generally to that which the entire body can accept. In many cases, it would be advantageous to be able to treat only a particular organ, or part of an organ.

Laparoscopic/endoscopic surgical instruments exist that allow a surgeon to see inside a body cavity of a patient without the necessity of large incisions. This reduces the chances of infection and other complications related to large incisions. The endoscope further allows the surgeon to manipulate microsurgical instruments without impeding the surgeon's view of the area under consideration. Although endoscopic surgical instruments are well developed and in use for surgical operations, an apparatus and method is not described or used in the prior art for delivering a treatment fluid interstitially to a precise target area within a body.

It is therefore apparent that there is a need for a method and apparatus that can deliver a treatment substance to the interior localized body area, and also a need for treatment formulations that are most effective when applied directly to a particular body part.

### SUMMARY

It is therefore an object of the present invention to provide an improved method for treating disease in a particular body part.

It is a further object of the present invention to provide a method of directly applying a treatment substance to a particular body part.

It is a still further object of the present invention to provide a specific formulation for effective treatment of each of various body parts.

It is another object of the present invention to provide specific compositions and concentration of gel formulations for treatment of corresponding body organs.

It is an object of the present invention to provide apparatus for injection of treatment substances in the form of a gel.

It is a further object of present invention to provide a method and apparatus for localized tissue treatment by injecting a treatment substance from the family of chemo-gels including ETOH gel, saline gel, acetic acid gel, biological gel, chemotherapeutic gel, polymer gel, protein gel, virus, genes and vector gel, antibiotic gel, energy activated gel, magnetic gel, contrast agent gel, photosensitive gel and other gel treatment substances.

It is another object of the present invention to provide a method and apparatus for injection treatment for prostate and urological disorders including enlarged prostate, BPH, prostatitis, prostate cancer and bladder tumors, using viscous injectable treatment substances.

It is another object of the present invention to provide a method and apparatus for injection treatment of body organs including a liver, kidney, lung, adrenal gland, gallbladder, and G.I. tract using a viscous injectable treatment substance.

It is another object of the present invention to provide a method and apparatus for injection treatment of breast tumors, cysts and malignant tumors using a viscous injectable treatment substance.

It is another object of the present invention to provide a method and apparatus for treatment of excessive bleeding of the uterine cavity and other gynecological disorders using a viscous injectable treatment substance.

It is another object of the present invention to provide a method and apparatus for injection treatment wherein an injection device is inserted into target body tissue requiring treatment using biopsy guides or through a working channel of an endoscopic instrument, an endoscope, imaging apparatus or other surgical or diagnostic/imaging instrumentation.

It is another object of the present invention to position or guide the injection apparatus to target body tissue using minimally invasive or non-invasive imaging or guiding methods and apparatus including endoscopes, ultrasound, CT, MRI, X-ray and other needle guiding or positioning/locating methods and apparatus.

Briefly, the present invention includes a method and apparatus for treating disease by injecting a treatment substance directly into the diseased body part, and thereby leaving the remaining body parts relatively unaffected. Specific treatment substance formulations are provided for each of a plurality of body parts for specific diseases. The treatment substance formulations contain two principle parts including a preferred active treatment (therapy) substance, and a preferred inactive binding (carrier) substance for thickening the treatment substance such as a specific gel or viscous material for carrying the preferred active treatment (therapy) substance for a particular disease and body part. The method also provides preferred treatment substance dosages to be injected into each body part. Apparatus for injecting the treatment substance is also provided that can be used with endoscopic instruments.

### IN THE DRAWING

FIG. 1 is a chart illustrating the method of the present invention;

FIG. 2 is a list of treatment substances;

FIG. 3 is a list of binding/gelling agents;

FIG. 4 is a list of electrically conductive materials;

FIG. 5A is a chart specifying preferred treatment substance formulations, compositions and dosages for treating specific diseases of the prostate with ethanol;

FIG. 5B is a chart specifying preferred treatment substance formulations, compositions and dosages for treating specific diseases of the prostate with a saline chemo gel;

FIG. 5C is a chart specifying formulations and dosages of treatment substances for all prostate diseases;

FIG. 6A is a chart specifying preferred treatment substance formulations and dosages for treating various specific diseases and body organs;

FIG. 6B is a chart specifying preferred treatment substance formulations and dosages for treating various diseases and body organs;

FIG. 7A is a chart showing various elements of the injection methods, delivery and imaging guidance methods of the present invention for each of a plurality of body organs and related diseases;

FIG. 7B is a chart showing various elements of injection delivery and imaging guidance methods of the present invention for each of a plurality of body organs and related diseases;

FIG. 8 shows injection of each of a plurality of body organs;

FIG. 9 illustrates injection of a prostate;

FIG. 10 shows injections of a body part through use of a syringe and needle, and through use of an endoscope and gel injection apparatus;

FIG. 11A shows a transurethral device for injection of a treatment substance, wherein the device can be inserted through a cystoscope;

FIG. 11B is a more detailed illustration of features of the device of FIG. 11A;

FIG. 12 shows a needle injection device inserted into a working channel of an endoscope, the assembly of which can be used with either a rigid or flexible cystoscope or resectoscope;

FIG. 13 illustrates the use of a flexible cystoscope, guided through a urethra by ultrasound imaging for injecting a treatment substance to a prostate;

FIG. 14 illustrates accessing a prostate percutaneously, or transperinially guided by a grid, and an ultrasonic rectal probe for injection of a treatment substance with a syringe under imaging guidance.

FIG. 15 illustrates accessing a prostate transrectally with a biopsy device or with an transrectal ultrasound imaging probe with a working channel, or with a needle device through the rectum, guided by an ultrasonic rectal probe for injection of a treatment substance, and alternatively with the added application of RF energy.

FIG. 16 shows an ultrasound probe with a working channel for guiding a needle; and

FIG. 17 shows an ultrasound probe with an external needle guide.

### DETAILED DESCRIPTION OF THE PREFERRED EMBODIMENT

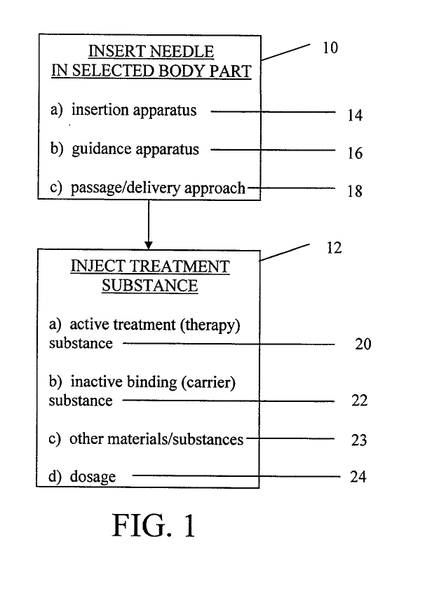

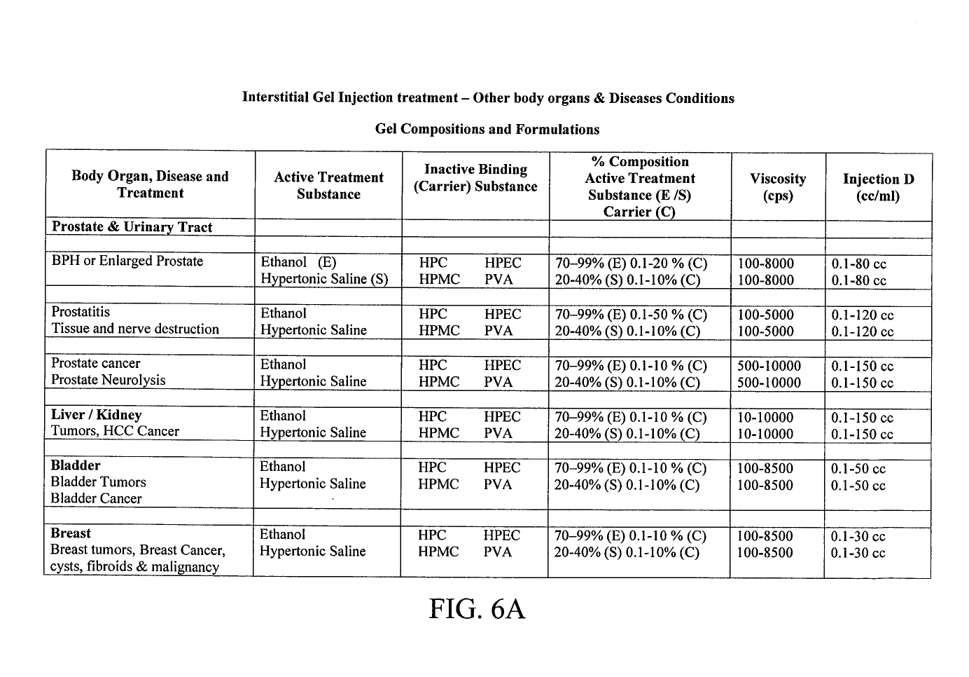

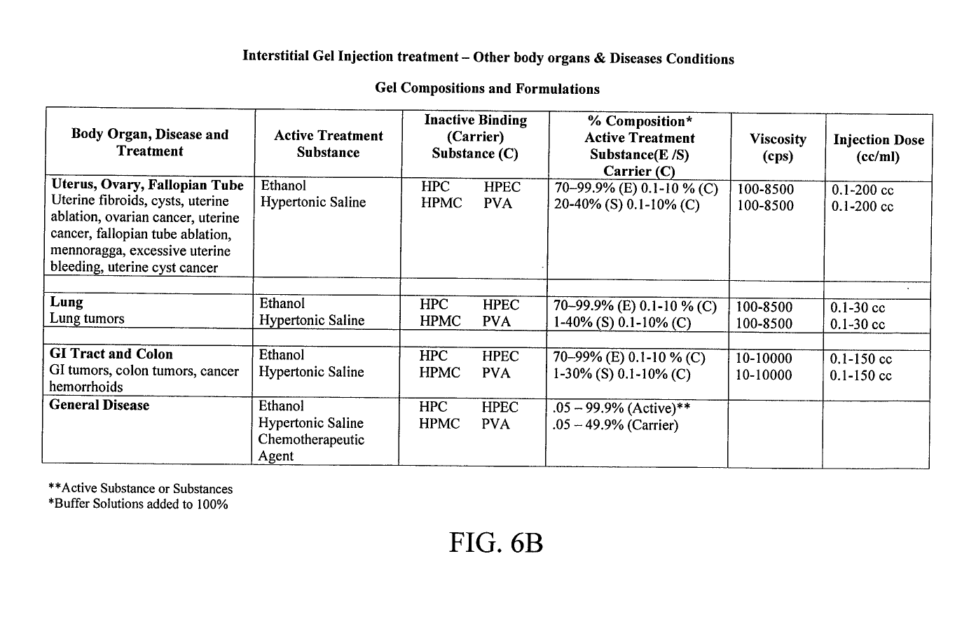

A preferred embodiment of the present invention will now be described in reference to the chart of FIG. 1 of the drawing. According to the method, a hollow core needle is inserted, by any of various methods and apparatus, into a person's body part to be treated (block 10). A treatment substance is then injected through the needle and into the body part (block 12), providing a localized application of the substance, leaving the remainder of the person's body relatively unaffected by the substance. The method applies generally to any disease treatable with an injected treatment substance, and any body part, including but not limited to a prostate, bladder, breast, uterus, lung, liver, kidney, fibroid, myoma, anus, gallbladder, adrenal gland and other body parts for example as listed in FIGS. 6A and 6B.

The methods and corresponding apparatus 14 for insertion of the needle include the use of any of a variety instruments, including a rigid or flexible endoscope, cystoscope, resectoscope, laparoscope and ultrasound probe. The insertion of the needle is guided by any of a variety of methods and apparatus 16, including but not limited to the scopes listed above, and including other invasive guidance apparatus, and non-invasive imaging methods and apparatus. The methods and apparatus can be, for example, an ultrasound probe, a needle guide, template, grid or other positioning and guiding apparatus. Non-invasive methods and apparatus for guiding the needle include ultrasound apparatus, CT, MRI, and X-ray apparatus.

The present invention includes bringing a needle to a selected body part by way of any selected body passage 18, such as through a urethra or rectum. The needle can also be brought to the selected body part through the skin, or through an incision in the skin and other means. For example, a needle can be brought to the prostate percutaneously through the skin of the perineal area or abdominal area, and then interstitially to the prostrate.

The injected treatment substance includes an active treatment (therapy) substance 20 and an inactive binding (carrier) substance 22 that carries the active treatment substance for controlling the dispersion of the treatment substance once injected into the body part. The active treatment substance 20 can be any material included for a particular active/treatment tissue effect. In addition to the carrier and active material, the substance can include other material 23 to provide required physical properties of the treatment substance for any non-active purpose. The treatment substance, for example, can include elements in any form, such as liquid, gas, solid, gel, viscous fluid, semi-liquid solutions, semisolid, suspensions, colloids, micro spheres and conjugates. For the purpose of generalization, the term “inactive binding substance” or “carrier” will be used to refer to both viscous and gel material. The inactive binding substance 22 slows the rate of dispersion, reducing the consequent volume of tissue treated by the active treatment substance and thereby increasing the concentration of the treatment substance in the body part for a given dosage 24. The concentration of the active substance in a body part depends in part on the viscosity of the inactive binding substance as discussed above, wherein a more viscous substance will disperse more slowly and therefore result in a higher concentration in a given volume of body part. The concentration of the active treatment substance also depends on the percentage of the active treatment substance in the treatment substance i.e., the ratio of the active substance to the inactive substance. These parameters will be discussed more completely in the text in reference to the following figures of the drawing.

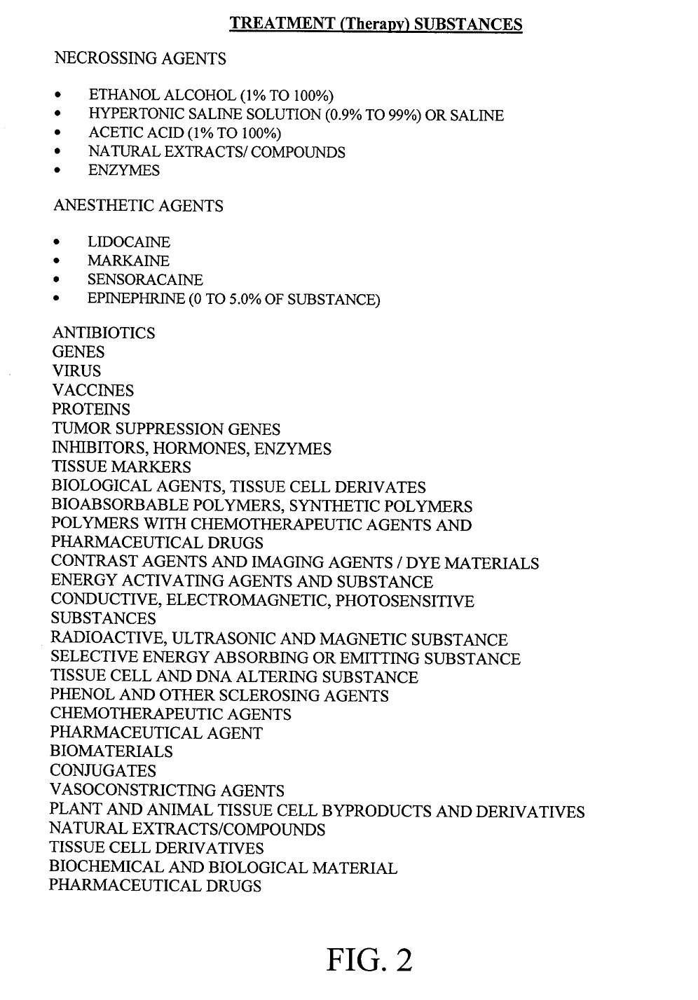

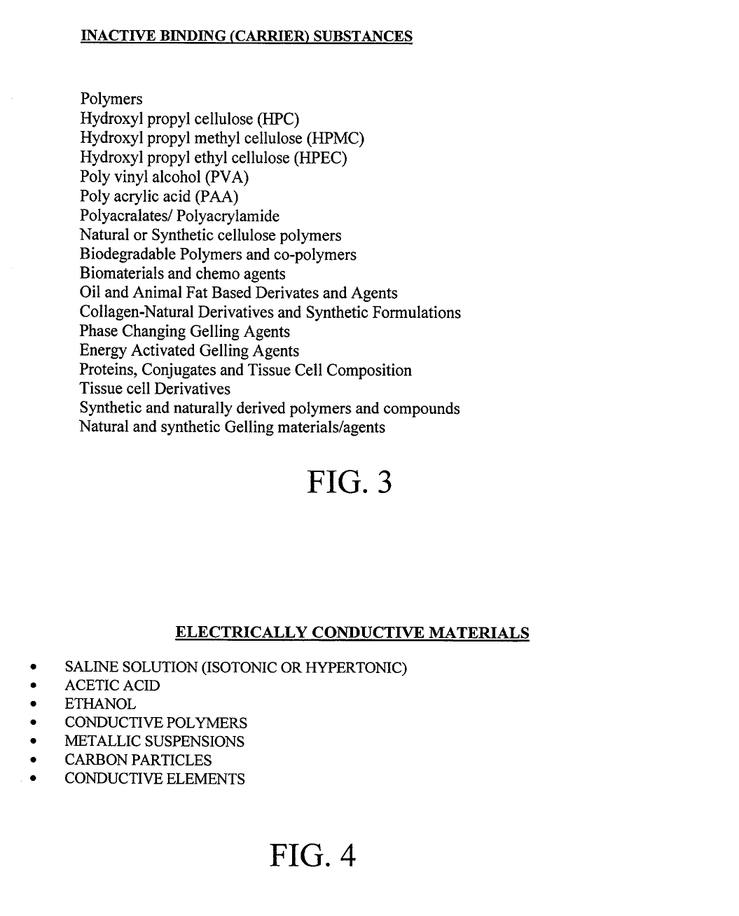

FIG. 2 is a list of active treatment substances, and FIG. 3 is a list of inactive binding (carrier) substances. One or more of the active treatment substances 20 of FIG. 2 and one or more of the inactive binding substances 22 of FIG. 3 may be selected and combined to form a treatment substance for injection. FIG. 4 is a list of electrically conductive substance that can also be added to the treatment substance or applied separately in the event that application of RF (radio frequency) energy is desired for treatment.

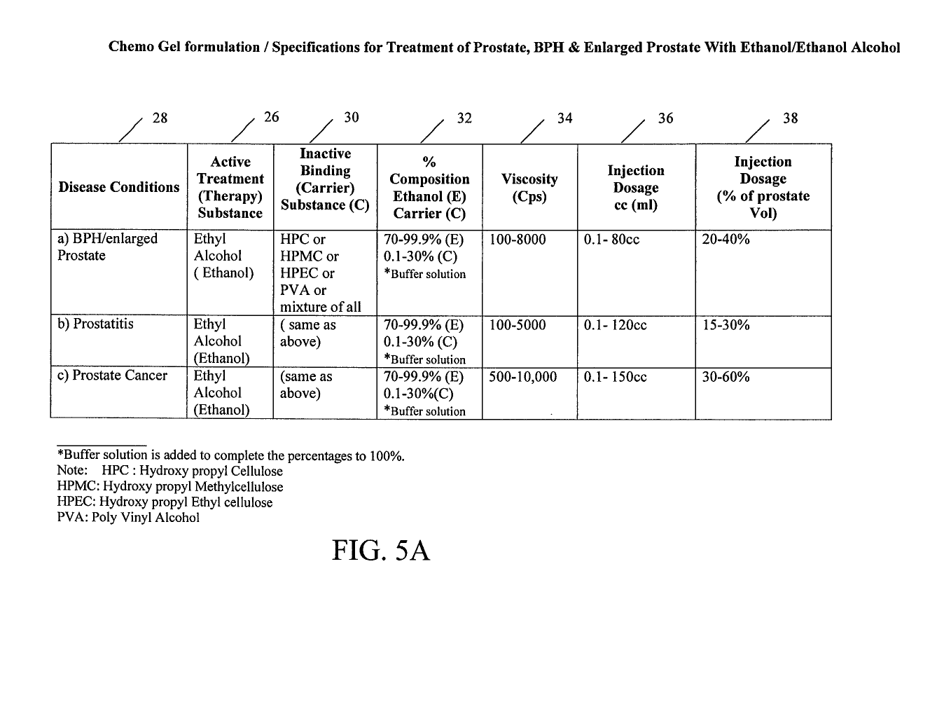

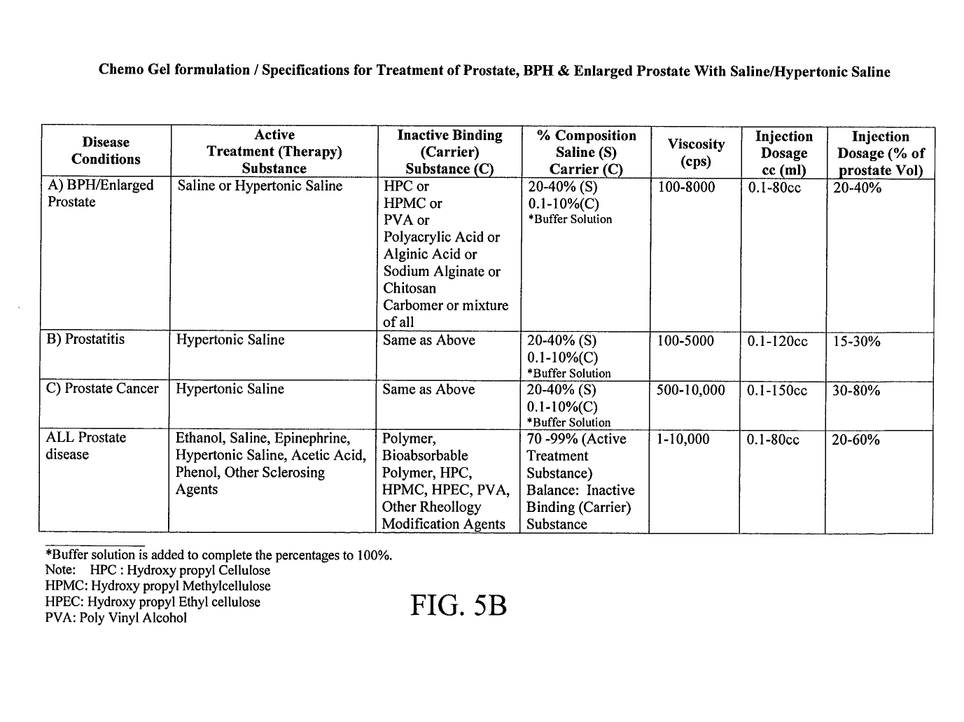

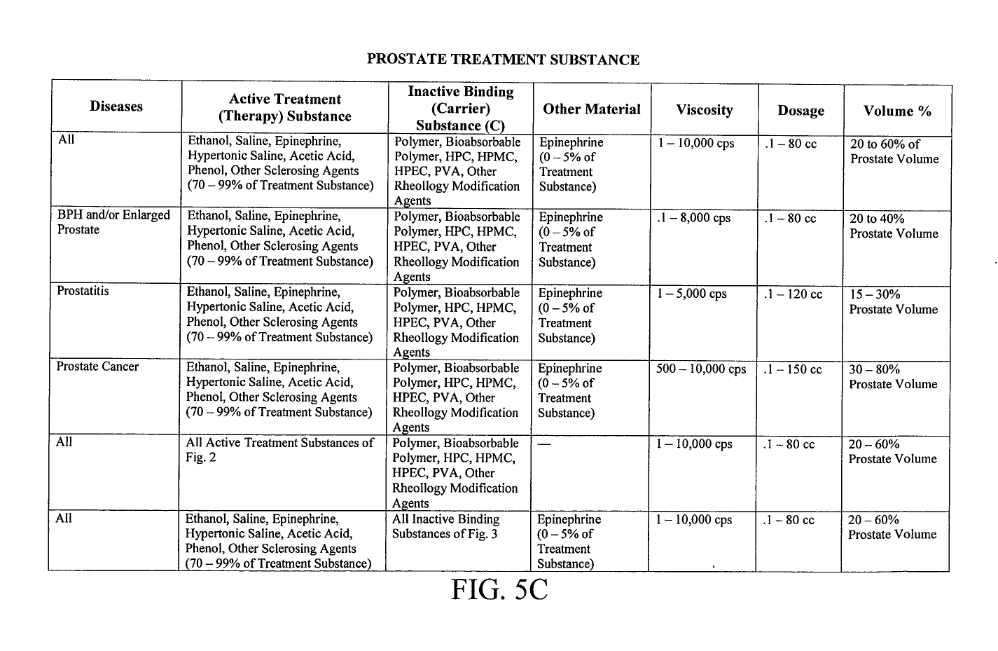

Specific treatment substance formulations for treating disease of the prostate are detailed in FIGS. 5A, 5B and 5C. FIG. 5A includes formulations using ethanol as the active substance, (column 26) for treatment of BPH/enlarged prostate, prostatitis, and prostate cancer (column 28). The inactive binding substance/carrier (gelling/viscous agent) is selected from the group listed in column 30, and includes the polymers HPC, HPMC, HPEC and PVA in any combination. The treatment substance includes the combination of at least one inactive substance and at least one active substance. The resultant treatment substance is indicated in column 32.

It should be noted in reference to FIGS. 5A-5C, and 6A and 6B that the percentages in the composition columns do not in all cases account for 100% of the treatment substance. In these cases, the remaining percentage is to be assumed to include a buffer solution such as water, etc. unless otherwise noted. For example, in the first row of FIG. 5A for treatment of BPH/enlarged prostate, column 32 shows a possible 70% ethanol, and a maximum carrier (C) substance of 20%. Since 70% plus 20% is only 90%, the balance of 10% is preferably a buffer substance such as water, but can also be another substance, either active or inactive as an alternate embodiment. This logic applies to all of the composition data in the various tables of the present specification. It should also be noted that although FIGS. 5A-5C and 6A and 6B show specific ranges for specific active and inactive substances, these are to be considered preferred embodiments, and other combinations are also included in the spirit of the present invention. For example, a single treatment substance may include more than one active substance, such as a combination of ethanol and saline solutions. FIG. 5C lists a variety of both active substances and inactive binding substances, and a treatment substance according to the present invention can include any combination of either the active substances and/or inactive substances.

Referring specifically to FIG. 5A as an example, for prostatitis the treatment substance includes an active substance of 70-99.9% ethanol, and 0.1-30% inactive binding (carrier) substance. The viscosity (column 34) for prostatitis is preferred to be in the range of 100-5000 cps. The injection dosage (column 36) for prostatitis is 0.1-120 cc, and in column 38 the injection dosage as a percentage of prostate volume is in the range of 15-30%. FIG. 5B is a chart for the preferred formulations for the same three prostate diseases as in FIG. 5A, except the active substance in the first three rows is saline or hypertonic saline for BPH/enlarged prostate, and hypertonic saline for prostatitis and prostate cancer. The fourth row formulation in FIG. 5B is a general formulation for all prostate diseases. FIG. 5C details further treatment substance formulations for treating prostate diseases. The treatment substances include at least one active treatment substance selected from the list in an amount equal to 70 to 99% of the treatment substance. The inactive binding materials are listed. Epinephrine is included in the formulation from 0 to 5% of the treatment substance. A general formulation for all prostate diseases is included in the first row listed in the table of FIG. 5C. The preferred viscosity is 1-10,000 cps, the dosage 0.1-80 cc, and the volume of prostate treated is in the range of 20-60%. FIG. 5C also includes treatment substance formulations for treatment of BPH and/or enlarged prostate, prostatitis, and prostate cancer, with the preferred viscosities, dosages and % volume treated. The formulation on the fifth row in FIG. 5C again refers to all prostate diseases, and includes one or more active substances selected from the list of FIG. 2. Similarly, the formulation of row six applies to all prostate diseases, and includes a selection of one or more inactive substances listed in FIG. 3.

FIGS. 6A and 6B list treatment substance formulations for use in treating specific diseases of body parts including a prostate, urinary tract, liver, kidney, bladder, breast, uterus, lung, G.I. tract and colon. In addition to the preferred specifications listed, FIG. 6B provides a formulation for treatment of any disease including 0.05-99.9% of an active substance such as ethanol, saline, or chemotherapeutic agent and 0.05-49.9% carrier inactive binding substance. The inactive binding (carrier) substance in FIG. 6B can be a polymer (P), or other material/substance to provide the required binding (gelling/viscous) carrier property desired. The general disease category of FIG. 6B includes treatment of diseases including vocal cord, pancreatic cancer, myomas, gastric tissue growth, gastric cancer and tissue growth, and any unspecified tumors and disorders, including the diseases of other organs listed in FIGS. 6A and 6B. The active treatment substance/chemotherapeutic agent, for example, can be for the purpose of tissue destruction.

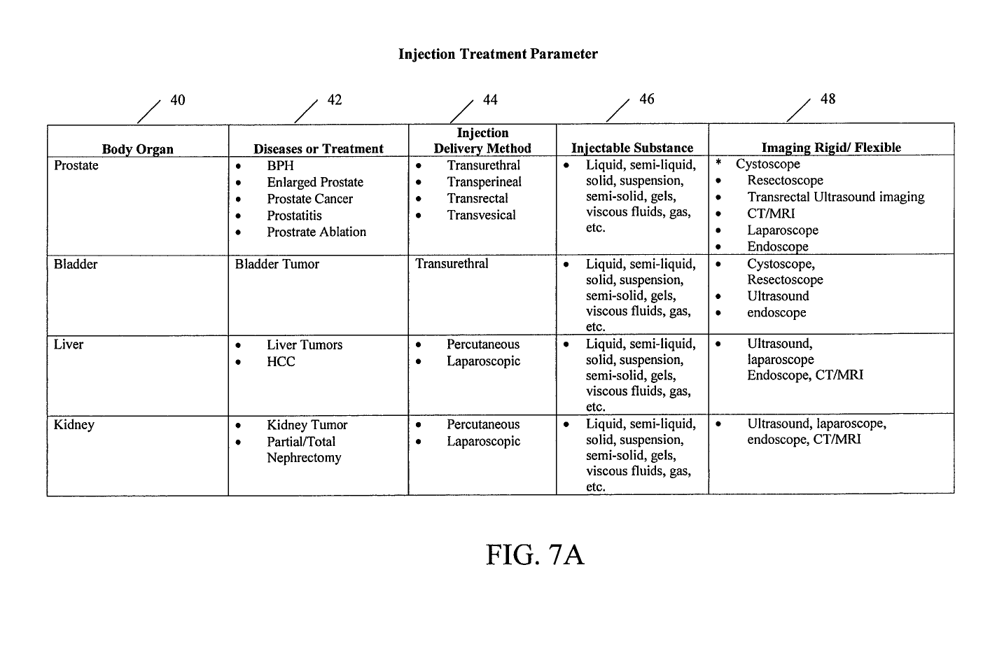

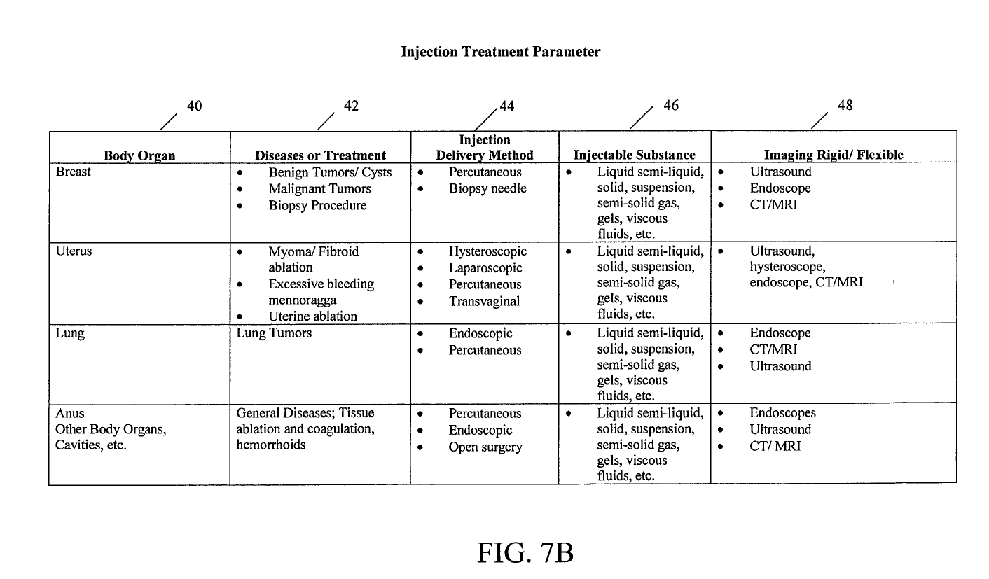

FIGS. 7A and 7B are a chart summarizing the method and apparatus for injection treatment of body parts including prostate, bladder, liver, kidney, breast, uterus, lung, anus, and other body organs. Column 40 indicates the particular body organ. Column 42 includes the diseases and/or treatment for each organ. Column 44 summarizes the passages through which apparatus including the hollow core injection needle are conveyed for delivering the needle to the particular organ. Column 46 lists various forms of elements that can be part of the treatment substance. Although FIG. 1 indicates that the preferred embodiment of the treatment substance includes an inactive substance/carrier that is a gelling/viscous agent in combination with an active substance, the form of the injected treatment substance can alternatively be any combination of the forms indicated in FIGS. 7A and 7B, including for example a solid and a gas. Column 48 lists apparatus that can be used in the process of delivering the needle to the selected body part. These lists include invasive and non-invasive guiding apparatus.

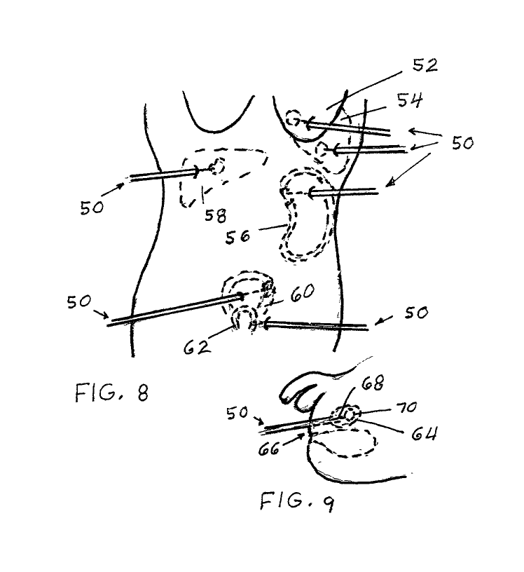

Application of the present invention is illustrated for various body parts in FIGS. 8 and 9, showing percutaneous access to parts of a body for injecting a treatment substance. Injection devices 50 are shown figuratively in FIG. 8 for treatment of a breast 52, lung 54, kidney 56, liver 58, uterus 60, and bladder 62. FIG. 9 shows an injection device 50 for percutaneously and interstitially accessing a prostate 64 through the perineal area 66, for insertion of a needle 68 to treat a target area 70 in the prostate 64.

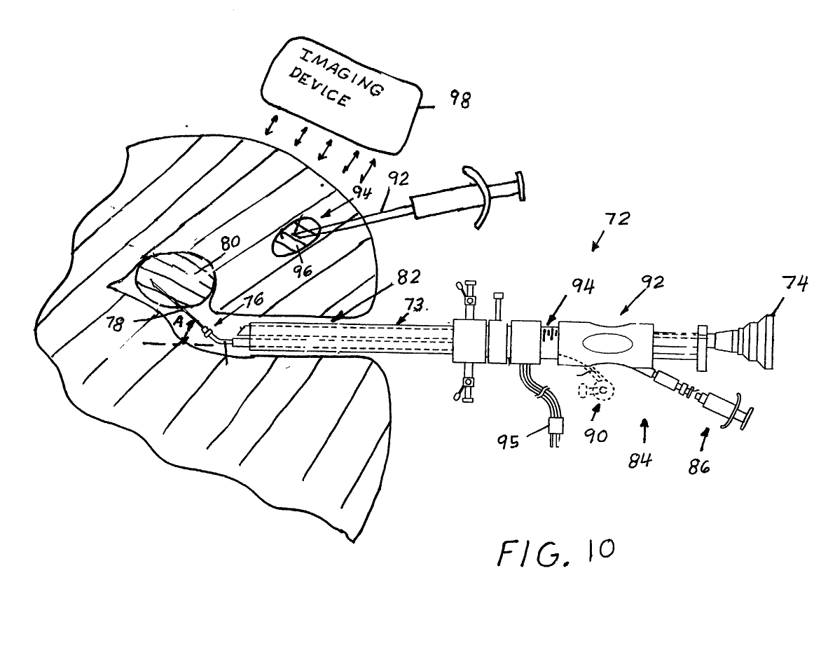

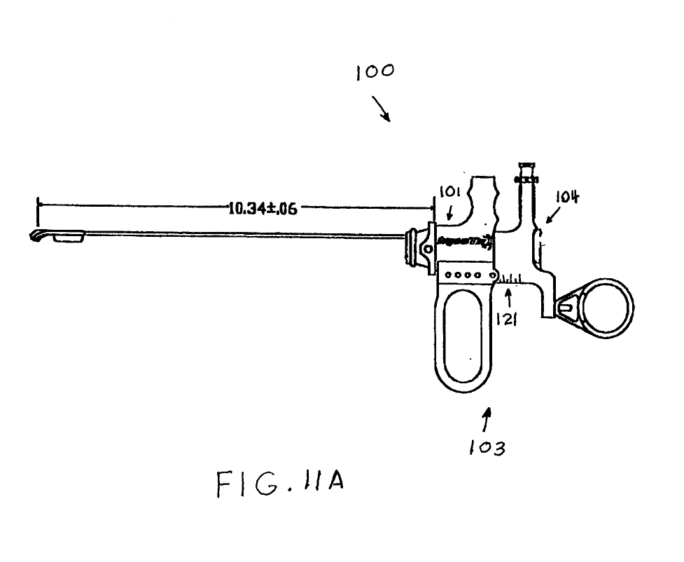

FIG. 10 shows the use of an endoscopic instrument 72 equipped with a scope 74 separately inserted through the instrument 72 housing 73 for viewing inside a body cavity 76 for visual guidance in directing a needle 78 into a target tissue 80. The endoscopic instrument 72 is shown inserted into a canal 82 which is representative in FIG. 10 of any body opening, natural or fabricated. The instrument 72 as shown includes a treatment substance injection apparatus 84, including the needle 78, and an injector 86, represented as a syringe type of device. The Instrument 72 includes a sliding mechanism 92 to move the needle 78 forward into the tissue 80. The needle path is controlled by visual marking 94 on the sliding mechanism handle. Furthermore the instrument 72 has an RF connector attachment 95 for application of RF energy. Further details relevant to the instrument 72, for example a device 90 for controlling the needle 78, are included in U.S. patent Ser. Nos. 09/510,537 and 09/715,853, the contents of which are incorporated in the present disclosure by reference. FIG. 10 also shows a needle 92 percutaneously inserted through interstitial tissue 94 to a target material 96. FIG. 10 symbolically illustrates guidance of the needle 92 to the target tissue 96 with a non-invasive imaging guidance device indicated by block 98. Those skilled in the art will know how to incorporate such apparatus 98 for the purpose of guiding the needle 92. The imaging device can be ultrasound, X-ray, MRI, CT, etc.

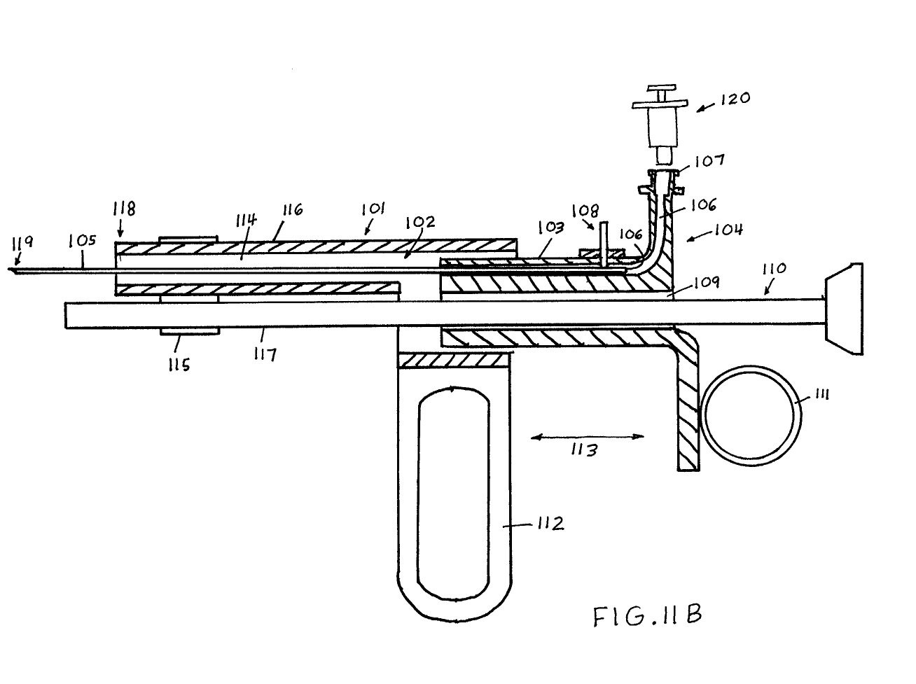

FIG. 11A is a scaled drawing that shows a transurethral injection device 100 designed to be used for treatment substance injection with a commercially available rigid cystoscope.

FIG. 1B is an enlarged and simplified cross sectional view of the device 100 of FIG. 11A. FIG. 11B is purposely not drawn to scale so that the various parts can be more clearly illustrated. The apparatus 100 is designed with a needle advancing mechanism included in first and second apparatus as follows. The first apparatus 101 has a channel 102 dimensioned for a sliding fit with the body 103 of the second apparatus 104. The second apparatus 104 has a hollow core needle 105 attached, with a proximal end 106 installed in fluid connection with a treatment substance channel 106 that can be fed by a treatment substance supply 120 connected to the channel 106 through a connector 107. The needle 105 is optimized for controlled delivery of a treatment substance including an inactive binding (carrier) substance as well as an active (therapy) substance as set forth in the various text and figures of the present specification. FIG. 11B also shows an electrical connection 108 in contact with the needle 105 for application of RF energy as an alternate embodiment, not shown in FIG. 11A. The second apparatus 104 also includes a channel 109 for passage of a cystoscope 110 as a separate device that can be used with the apparatus 100, and shown in FIG. 1I B for illustration. The second apparatus 104 includes a thumb ring 111 or other device for allowing an operator to move the second apparatus 104 relative to the first apparatus 101 by simultaneously gripping the ring 111 and slotted handle 112, allowing an operator to move the first apparatus relative to the second apparatus as indicated by the two way arrow 113. The first apparatus includes a needle guide channel 114 for passage of the needle 105, and a scope clip 115 for positioning the scope 110 relative to the first apparatus 101.

In operation, the tube 116 with needle 105 and optionally the scope probe 117 are inserted into a body passage such as a urethra. When the end 118 of the tube 116 is in the desired position near body tissue to be treated, an operator moves the needle 105 forward by compressibly gripping the ring 111 and handle 112, forcing the body 103 of the second apparatus 104 into the channel 102 of the first apparatus, driving the needle tip 119 into the desired tissue. The operator then activates a treatment substance source 120, such as a syringe attached to the connector 107, to drive the treatment substance through and out of the needle 105 into the diseased tissue. The position of the tube end 118 and needle tip 119 can be observed either through use of the scope 110 or through use of a non-invasive imaging device such as illustrated in FIG. 10, or a combination of the two methods. The depth of penetration of the needle can be monitored through use of the imaging device and/or through use of calibration marks on the apparatus 100, such as at 121, indicating the relative positions of the first and second apparatus 101 and 104. For assistance in non-invasive imaging of the needle position, the area of the tip 119 is alternatively constructed to include echogenic material as also discussed elsewhere in the present disclosure.

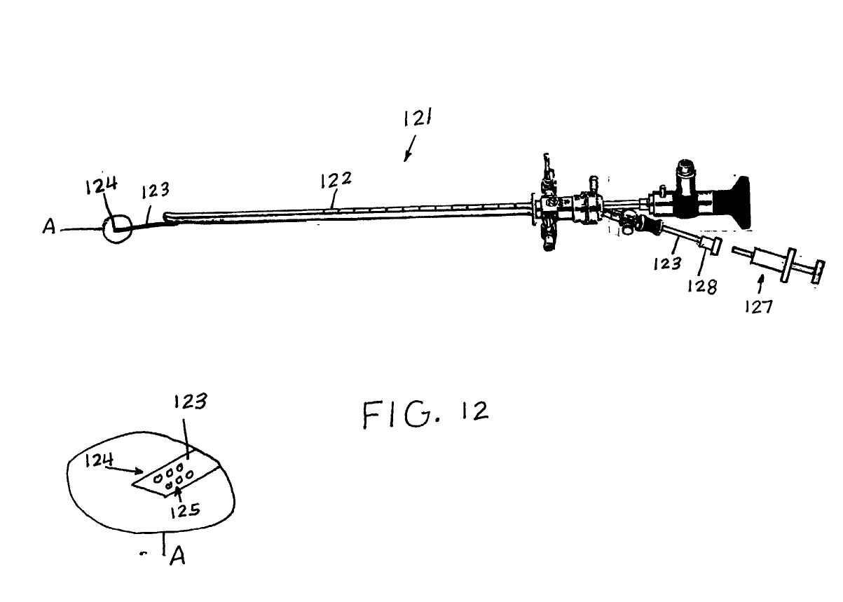

FIG. 12 is a view of an endoscopic apparatus 121, similar to the apparatus 72 of FIG. 10, except the relative dimensions are correctly shown for an actual working transurethral apparatus, but not drawn for ease of illustration of the various parts. For a detailed description of the working apparatus, refer to FIG. 10 and the corresponding description. FIG. 12 shows the transurethral apparatus 121 as having a long, slender tube 122, which can be either rigid or flexible. The apparatus 121 includes an injection needle 123 configured (length and diameter) for optimum injection of a treatment substance having an inactive binding (carrier) substance and an active treatment substance as described in the various figures of the present disclosure. As described in reference to the similar device of FIG. 10, the injection needle 123 can be deployed manually under endoscopic visualization for injection treatment, and/or can be guided an imaging method as described above. In this case, the injection needle tip 124 is designed for high echogenecity, and as shown in the expanded Section A, with one or more holes 125 with various sizes and patterns for optimum distribution of the treatment substance for a desired tissue effect. The injection needle can also be made from super elastic materials for curved or angular tip articulation. FIG. 12 shows a treatment substance source 126, illustrated symbolically as a syringe 127 for connection to the needle by way of connector 128.

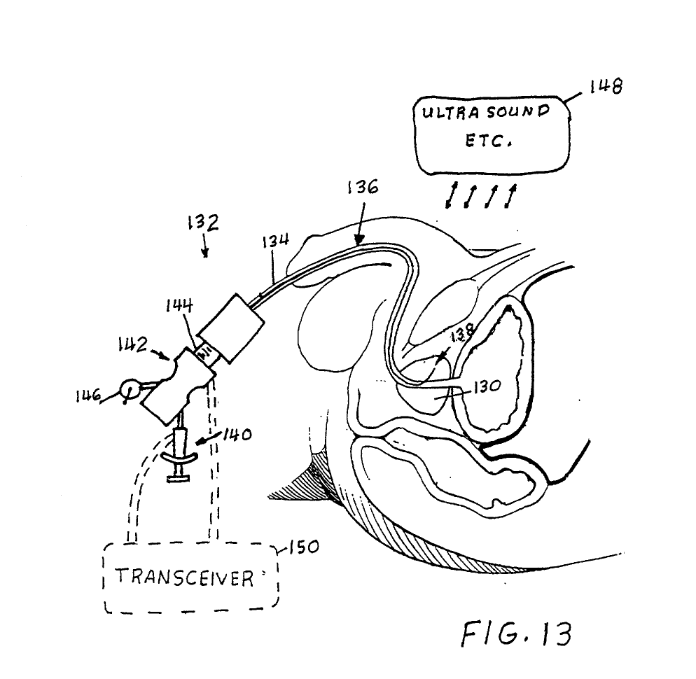

Transurethral access to a prostate 130 is illustrated in FIG. 13. A urological instrument 132 probe 134, which can be either rigid or flexible, is inserted into the urethra 136. One or more hollow core needles 138 are inserted through a working channel of the probe 134. The urological instrument can be, for example, a rigid or flexible cystoscope, resectoscope, endoscope, etc., and can be a special/novel design, or any of a variety of commercially available instrumentation. The instrument 132 has a substance injection device 140. The depth of the needle 138 is controllable by an adjustment device 142 and scale 144. A needle curvature adjustment apparatus is symbolically represented by item 146. Further details of these features of the device 132, including ultrasound imaging device 148 and transceiver 150 are described in U.S. patent application Ser. No. 09/510,537 incorporated by reference.

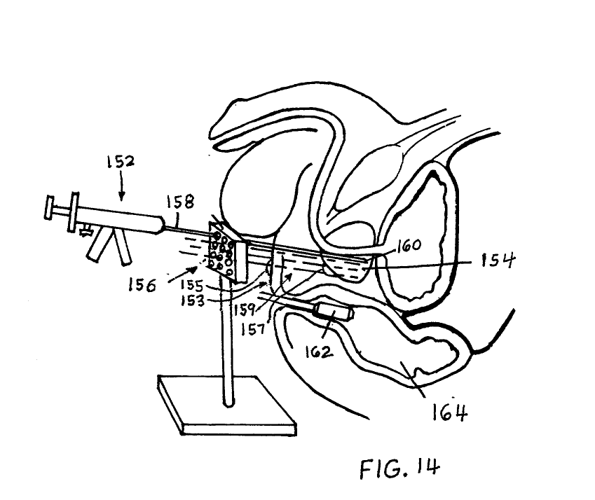

A transperineal device 152 for percutaneous access to a prostate 154 is illustrated in FIG. 14. The transperineal device is designed to be used independently or in conjunction with guide templates, grids, guides 156, or other positioning/guiding apparatus. FIG. 14 is convenient to illustrate that needle passage can proceed through various tissue types. The needle 158 of FIG. 14 can pass through skin 153, vesicles 155, 157 and interstitial space 159. Alternatively, the transperineal injection needle 158 device, preferably 20-14 gauge size also preferably has an echogenic tip 160, a feature that is desirable for injection as applied also to all other body organs, and is designed to inject a treatment substance in the form of a gel and/or a viscous substance as discussed in the various figures and text of the present disclosure into the prostate 154 under ultrasound imaging guidance, symbolized by an ultrasonic probe 162 in the rectum 164. The injection device needle tip 160 can be straight, curved, angular or articulating to inject any part of the prostate anatomy or lower urinary tract. The injection needle device 152 and positioning/guiding template 156 can be designed to allow semi-automatic or automatic injection treatment operation and a programmed dosage plan using computer software and a needle advancement and retraction mechanism. The treatment substance of the present invention can alternatively include in addition to the materials described above, an element providing a hyper echoic characteristic, making it visible under ultrasound, CT or MRI imaging. The actual location of injectable treatment substance in the target tissue and extent of volumetric coverage in situ can be monitored on a “real time” basis using the transrectal ultrasound probe 162. The injection needle tip 160 and treatment substance are visible as a bright white echogenic reflection, which can be controlled by adjusting the injection dosage volume in an interactive mode. The transitional zone of a prostate for BPH treatment can be targeted by injecting a treatment substance including an active substance, and an inactive binding substance; i.e., a thickened carrier (gel or viscous) substance under ultrasound imaging guidance. The typical injection dosage of treatment substance for BPH treatment varies between 10-45% of prostate volume measured by TRUS (Transrectal Ultrasound). The use of a treatment substance with a hyper echoic property and/or a hyper echoic needle tip, visible under non-invasive imaging applies as an element in an alternate embodiment for injection of any organ or method as described in the present disclosure.

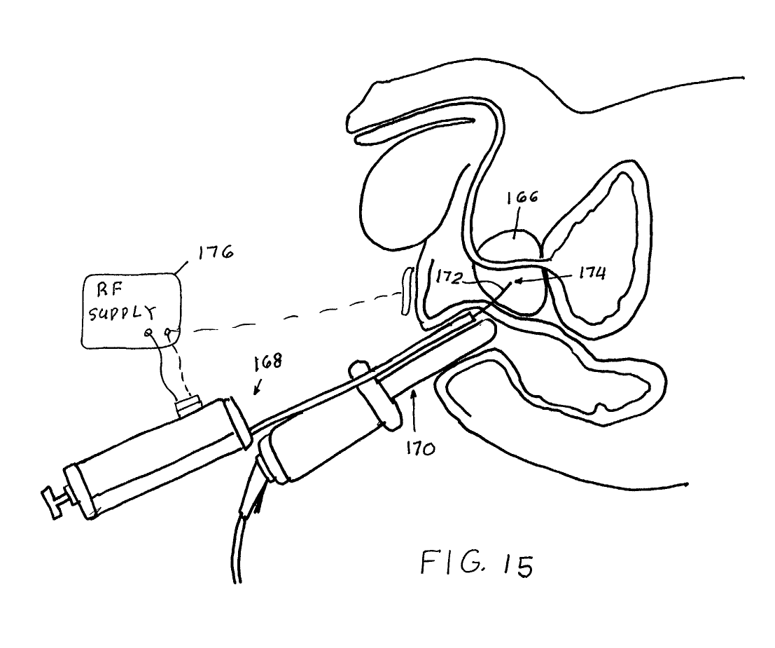

Transrectal access to a prostrate 166 is illustrated in FIG. 15. A transrectal injection device 168 is designed to be used independently or in conjunction with a transrectal ultrasound probe 170, or a transrectal biopsy probe or transrectal MRI probe. The transrectal injection needle device (20-14 ga size) 172 preferably has an echogenic tip 174 and is designed to inject a treatment substance as described in the above text and figures of the drawing into the prostate 166 under ultrasound imaging guidance. The injection device needle tip 174 can be straight, curved, angular or articulating to inject any part of the prostate anatomy or lower urinary tract. The injection device 158 can be designed to allow semiautomatic or automatic injection treatment operation. The echogenic injection needle tip and treatment substance are visible as bright white echogenic reflections, which can be controlled by the volumetric dosage of the treatment substance. A commercially available biopsy needle probe can also be used to inject the treatment substance under TRUS ultrasound imaging guidance. The treatment substance can be injected through a hollow core needle of the biopsy needle probe. The injection needle can be inserted through a working channel of a transrectal ultrasound probe or through a biopsy needle guide mounted on a TRUS probe. A typical dosage of treatment substance for treatment of an enlarged prostate or BPH varies between 10-45% of prostatic volume; as measured by TRUS. FIG. 15 also shows an RF supply 176 for application of RF (radio frequency) energy to the prostate. This is described in more detail in U.S. patent Ser. No. 9/510,537.

The preferred injectable treatment substance used for intraprostatic injection treatment of enlarged prostate and BPH consists of a family of chemo-gels and viscous injectable formulations including; ethanol gels, saline gels, biological gels, chemotherapeutic gels, bioabsorbable gels, polymer gels, pharmaceutical gels and other proprietary gels and viscous substance formulations. The treatment substance consists of an aqueous, viscous composition of an active treatment substance and an inactive binding (carrier) substance, and may also include other complimentary chemical agents including for example a buffer substance, and/or epiniphrine and/or an echo-genic substance, etc. as required. The inactive binding substance provides appropriate viscosity to the treatment substance as explained above. The composition, molecular weight and concentration of the active treatment substance in relation to other agents and additives can determine the physical and chemical properties of the treatment substance. The treatment substance formulation is preferably hyperechoic so as to be readily visible under ultrasound, CT and MRI imaging. A treatment substance with a visible characteristic allows real time, interactive control during injection treatment by varying the dosage volume. The active treatment substance portion of the treatment substance, its concentration, specification and physical properties are designed to create an optimum therapeutic effect in prostatic tissue for treatment of various prostatic diseases and urological disorders.

Based on extensive clinical studies and patient follow-up evaluation, detailed specifications and physical properties for various treatment substance formulations have been established for treatment of enlarged prostate, BPH, prostate cancer, bladder cancer and other urological disorders. The treatment substance formulation and optimum injection volume dosage have also been established for treatment of various prostate diseases. The detailed formulations for treatment substances are outlined in FIGS. 5A and 5B for prostate, and FIGS. 6A and 6B for a variety of organs.

The percutaneous and interstitial injection treatment using a treatment substance (illustrated in FIGS. 8 and 9) has potential application for diseases of various body organs including breast, uterus, lungs, liver, kidney, myomas, fibroids, anus, adrenal gland, gallbladder, etc. The controlled tissue ablation in a body organ or body cavity can be accomplished with the method and apparatus described above for treatment of prostate and bladder cancer, BPH, prostatitis, breast cancer, uterine fibroids and cavity ablation, lung, liver, kidney tumors and various other diseases.

The viscous treatment substance creates a localized desirable effect in the target tissue without causing undesirable side effects in surrounding body organs or a systemic effect in the entire body. The treatment substance specification for a specific disease treatment includes its composition, % concentration, physical properties including viscosity, molecular weight and specific gravity, along with an appropriate dosage level for an optimum clinical outcome. The general list of various active treatment substances and inactive binding (carrier) substances used for injection treatment are outlined in FIGS. 2, 3 and 4. Furthermore, a specific formulation, specification and dosage level for treatment of each specific disease indication is outlined in FIGS. 6A and 6B. The injectable treatment substance formulations, specifications and dosage levels for treatment of prostate diseases including BPH/enlarged prostate, prostatitis, prostate cancer are listed in FIGS. 5A and 5B.

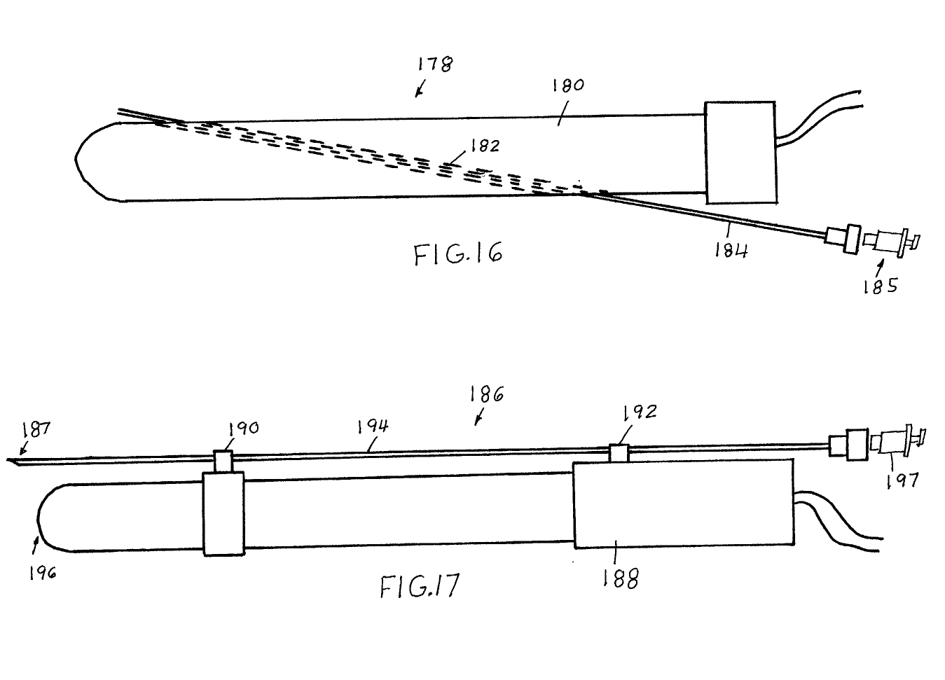

FIG. 16 shows a combination needle guide and ultrasonic ultrasound probe apparatus 178 including a functional ultrasound probe portion 180 for imaging, and a channel 182 built into the probe apparatus 178 for guiding a hollow core needle 184, wherein the needle 184 is configured for injecting a treatment substance as disclosed above.

In operation, the needle 184 is retracted so as to place the tip 184 inside the channel 182. The probe 180 is then inserted into a body passage, such as a rectum. When the operator observes via the ultrasound imaging that the probe is placed as required for insertion of the needle 184 into a diseased tissue, the needle is thrust forward into the tissue to the desired depth, which can be observed through use of the ultrasound imaging apparatus. The treatment substance is then propelled through the needle 184 by use of a propulsion device symbolically illustrated by syringe 185.

FIG. 17 shows a combination needle guide and ultrasound probe apparatus 186, including an ultrasound probe apparatus 188 for imaging, and an attached guide apparatus (190, 192) for guiding a hollow core needle 194 along the outside of the probe 188. In commercially available equipment, guide apparatus such as 190 and 192 is provided for guiding a biopsy needle. According to the present invention, this biopsy needle guide apparatus is used to guide the needle 194 configured for injection of a treatment substance. The operation of the apparatus 186 involves first placing a protective covering (condom) over the needle tip 187, with the needle in a withdrawn position behind the tip 196 of the probe 188. Alternatively, the needle tip can be retracted within a structure such as guide support 190, and thereby also preventing the needle tip from penetrating body tissue while the probe and needle assembly 186 is being positioned within a body passage. The probe and needle apparatus 186 is then inserted into a body passage such as a rectum. With the probe tip 196 in the desired position for inserting the needle 194, the needle 194 is thrust forward, through the protective covering (not shown), and into the desired tissue (not shown) to the desired depth, which can be monitored by an ultrasound imaging apparatus including the probe and related instrumentation not shown. The treatment substance is then propelled through the needle 194 by a propulsion device 197 symbolically illustrated as a syringe.

In summary, the present invention relates generally to methods and apparatus for injection treatment of various injectable treatment substances, including the substance concentrations, compositions, formulation and other physical properties to achieve optimum parameters for treatment of BPH and prostate disorders, and diseases associated with other body organs. The injection treatment substance is injected into a prostate and other body organs in the form of a gel or highly viscous substance for a controlled therapeutic or tissue effect. The viscous gel formulation of the injectable treatment substance creates a localized tissue effect in the target area without causing undesirable side effects in surrounding organs or throughout the patient's body. The gel formulation is injected into a diseased body portion through use of any one of various devices known to those skilled in the art. This was illustrated in FIG. 8 figuratively illustrating injection devices 50, which can be applied to any organ as required. A laparoscope or endoscope device, known to those skilled in the art, can be inserted through an incision for use in guiding an injection needle to a target tissue, such as the liver, kidney, uterus, bladder, breast or lung, or other organ. In guiding a needle to a precise target, the optics of a laparoscope or other similar device is often helpful. The use of a non-invasive ultrasound imaging technique is also included in the spirit of the present invention for guiding a needle. This is helpful in guiding a biopsy device, and can also be used as an additional optional aid when using an endoscope or similar device.

Although the present invention has been described above in terms of a specific embodiment, it is anticipated that alterations and modifications thereof will no doubt become apparent to those skilled in the art. It is therefore intended that the following claims be interpreted as covering all such alterations and modifications as fall within the true spirit and scope of the invention.

What is claimed is:

1. A method for treating a portion of a body part comprising:
a) inserting a needle apparatus in said body part, said apparatus including at least one hollow core needle for delivering a treatment substance into said body part, said treatment substance including an active treatment substance, and an inactive binding substance;
b) guiding said needle apparatus to target tissue in said body part in need of treatment; and
c) injecting said treatment substance to said target tissue through said needle apparatus.

2. A method as recited in claim 1 wherein said treatment substance is further formulated so as to be visible in real time under non-invasive imaging selected from the group consisting of CT, MRI, x-ray and ultrasound.

3. A method as recited in claim 1 wherein said body part is a prostate.

4. A method as recited in claim 3 wherein said active treatment substance is ethanol and said inactive substance is a polymer gelling agent.

5. A method as recited in claim 4 wherein said active treatment substance is ethanol in an amount equal to 70 to 99.9 percent of said treatment substance, and said inactive substance is a polymer in an amount equal to 0.1 to 20 percent of said treatment substance, and wherein said treatment substance is for treatment of BPH/enlarged prostate.

6. A method as recited in claim 4 wherein said active treatment substance is ethanol in an amount equal to 70 to 90 percent of said treatment substance, and said inactive substance is a polymer in an amount equal to 0.1 to 50 percent of said treatment substance.

7. A method as recited in claim 4 wherein said active treatment substance is ethanol in an amount equal to 70 to 99 percent of said treatment substance, and said inactive substance is in an amount equal to 0.1-10 percent of said treatment substance.

8. A method as recited in claim 3 wherein said active treatment substance is saline and said inactive substance is a polymer.

9. A method as recited in claim 8 wherein said active treatment substance is saline in an amount equal to 20 to 40 percent of said treatment substance, and said inactive substance is in an amount equal to 0.1 to 10 percent of said treatment substance, and said treatment substance is for treatment of prostate diseases selected from the group consisting of BPH/enlarged prostate, prostatitis and prostate cancer.

10. A method as recited in claim 4 wherein said inactive substance is a polymer with one or more constituents selected from the group consisting of HPC, HPMC, HPEC and PVA.

11. A method as recited in claim 8 wherein said inactive substance is a polymer with one or more constituents selected from the group consisting of HPC, HPMC, PVA, polyacrylic acid, alginic acid, sodium algenate and chitosan carbomer.

12. A method as recited in claim 3 wherein said inserting includes passage of said needle apparatus percutaneously and interstitially to said prostate through a perineal area.

13. A method as recited in claim 3 wherein said inserting includes passage of said needle apparatus through a urethra.

14. A method as recited in claim 3 wherein said inserting includes passage of said needle apparatus through a rectum.

15. A method as recited in claim 5 wherein
a) said treatment substance has a viscosity property in the range of 100 to 8,000 cps; and
b) an injection dosage in the range of 0.1 to 80 cc.

16. A method as recited in claim 6 wherein
a) said treatment substance has a viscosity in the range of 100 to 5,000 cps; and
b) an injection dosage in the range of 0.1 to 120 cc.

17. A method as recited in claim 7 wherein
a) said treatment substance has a viscosity in the range of 500 to 10,000 cps; and
b) an injection dosage in the range of 0.1 to 150 cc.

18. A method as recited in claim 9 wherein for treatment of said BPH/enlarged prostate
a) said treatment substance has a viscosity in the range of 100-8,000 cps; and
b) an injection dosage is in the range of 0.1 to 80 cc.

19. A method as recited in claim 9 wherein for treatment of said prostatitis
a) said treatment substance has a viscosity in the range of 100-5,000 cps; and
b) an injection dosage is in the range of 0.1 to 120 cc.

20. A method as recited in claim 9 wherein for treatment of said prostate cancer
a) said treatment substance has a viscosity in the range of 500-10,000 cps; and
b) an injection dosage in the range of 0.1 to 150 cc.

21. A substance injection device comprising:
a transurethral apparatus including
a) a first apparatus having
i) a housing with a first channel, and an elongated tube extending from said first channel, and a handle configured for push-pull operation; and
b) a second apparatus having
i) a body with an elongated portion configured for a sliding fit in said first channel, and a hollow core needle extending from said body so as to be guided by said elongated tube, and a treatment substance input apparatus for forcing said treatment substance through said needle, and a ring wherein an operator can move said needle forward or backwards within said tube by an operator using said handle and said ring.

22. A device as recited in claim 21 wherein said second apparatus further includes an RF input connector for applying RF to said needle.

23. A method for treating a portion of a body part comprising:
a) inserting a needle apparatus in said body part, said apparatus including at least one hollow core needle for delivering a treatment substance into said body part, said treatment substance including an active treatment substance, and an inactive substance;
b) guiding said needle apparatus to target tissue in said body part in need of treatment including use of an imaging device; and
c) injecting said treatment substance to said target tissue using said needle apparatus;
wherein said treatment substance includes one or more substances selected from the group consisting of chemo-therapeutic agents and sclerosing agents, including ethanol, saline, acidic agents, epinephrine, biological agents, polymers, bioabsorbable polymers, proteins, conjugates plants, animal tissue derivates and byproducts and pharmaceutical drugs.

24. A method for treating a portion of a body part comprising:
a) inserting a needle apparatus in said body part, said apparatus including at least one hollow core needle for delivering a treatment substance into said body, said treatment substance an inactive binding substance and an active treatment substance;
b) guiding said needle apparatus to target tissue in said body part in need of treatment including use of an imaging device; and
c) injecting said treatment substance into said target tissue using said needle apparatus;
wherein the treatment substance includes a material selected from the group consisting of liquids, gases, solids, gels, viscous fluids, semi-liquid solutions, semi-solids, suspensions, colloids, micro spheres and conjugates.

25. A method for treating a portion of a body part comprising:
a) inserting a needle apparatus in said body part, said apparatus including at least one hollow core needle for delivering a treatment substance into said body, said treatment substance including an inactive binding substance and an active treatment substance;
b) guiding said needle apparatus to a target tissue in said body part in need of treatment including use of an imaging device; and
c) injecting said treatment substance into said target tissue using said needle apparatus;
wherein said treatment substance includes an element selected from the group consisting of chemo-therapeutic agents, sclerosing agents, ethanol, saline, acetic acid injectable agents, anesthetic agents, antibiotics, enzymes, biological agents, bioabsorbable polymers, biomaterials, conjugates, pharmaceutical drugs, genes, viruses, vasoconstricting agents, proteins, contrast agents, polymers, plant and animal tissue cell byproducts and derivatives, natural extracts/compounds and other biochemical agents.

26. A method for treating a portion of a body part comprising:
a) inserting a needle apparatus in said body part, said apparatus including at least one hollow core needle for delivering a treatment substance into said body part, said treatment substance including an inactive binding substance and an active treatment substance.
b) guiding said needle apparatus to a target tissue in need of treatment including use of an imaging device; and
c) injecting said treatment substance into said target tissue;
wherein said treatment substance is formulated with physical properties and specifications designed for treatment of specific body tissue and body organs selected from the group consisting of prostate, bladder, uterus, breast, vocal cords, gallbladder, pancreas, lungs, anus, colon, G.I. tract, kidney, liver and other body organs, body cavities and glands.

27. A method for treating a portion of a body part comprising:
a) inserting a needle apparatus in said body part, said apparatus including at least one hollow core needle for delivering a treatment substance into said body, said treatment substance including an inactive binding substance and an active treatment substance;
b) guiding said needle apparatus to a target tissue in need of treatment including use of an imaging device; and
c) injecting said treatment substance into said target tissue;
wherein said treatment substance is designed for treatment of a specific disease condition selected from the group consisting of BPH, enlarged prostate growth, prostatitis, prostate cancer, bladder tumors, breast tumors, cysts, breast cancer, pancreatic cancer, uterine fibroids, myomas, excessive uterine bleeding, uterine cyst cancer, lung tumor, hemorrhoids, gastric tissue growth, gastric cancer and tissue growth, tumors and disorder.

28. A method for treating a portion of a body part comprising:
a) inserting a needle apparatus in said body part, said apparatus including at least one hollow core needle for delivering a treatment substance into said body, said treatment substance including an inactive binding substance and an active treatment substance;
b) guiding said needle apparatus to a target tissue in said body part in need of treatment including use of an imaging device; and
c) injecting said treatment substance into said target tissue;
wherein said inserting includes passing said needle apparatus through a portion of said body selected from the group consisting of skin, interstitial material, rectum and urethra.

29. A method for treatment of a prostate comprising:
a) inserting an injection needle apparatus in a prostate, said apparatus including at least one hollow core needle for delivery of a treatment substance into said prostate, said treatment substance including an inactive binding substance and an active treatment substance, and said injection needle apparatus including instrumentation selected from the group consisting of a biopsy needle guide, and a working channel of an endoscope;
b) guiding said injection needle apparatus through a body passage selected from the group consisting of skin, urethra, rectum, perineum, and vesicle for treatment of a disease selected from the group consisting of BPH, enlarged prostate, and prostate cancer, said guiding using a method employing instrumentation selected from the group consisting of cystoscope, resectoscope, endoscope, and noninvasive image apparatus including transrectal ultrasound imaging apparatus, CT, MRI, and X-ray imaging apparatus; and
c) injecting said treatment substance into said prostate through said injection apparatus;
wherein said treatment substance includes elements selected from the group consisting of chemo-gels, including ethanol gel, saline gel, acetic acid gel, antibiotic gels, anesthetic gel, contrast agent gels, bioabsorbable polymer gel, epinephrine gel and tissue cell derivatives, chemo-therapeutic and other biochemical, polymer and biological gels.

30. A method for treating a localized portion of a prostate comprising:
a) inserting an injection needle apparatus into a prostate, said apparatus including at least one hollow core needle for delivery of a treatment substance into said prostate, said treatment substance including an inactive binding substance and an active treatment substance;
b) guiding said injection needle apparatus through a passage selected from the group consisting of a urethra, rectum, perineum, and vesicle, into said prostate for treatment of a disease selected from the group consisting of BPH, enlarged prostate and prostate cancer, using instrumentation selected from the group consisting of a cystoscope, resectoscope, endoscope, and non-invasive imaging apparatus selected from the group consisting of transrectal ultrasound imaging apparatus, CT, MRI, and x-ray apparatus; and
c) injecting said treatment substance into said prostate through a said injection apparatus, selected from the group consisting of a biopsy needle guide, working channel of an endoscope, and ultrasound imaging probe;
wherein said treatment substance includes elements selected from the group consisting of a chemo-gel formulation, sclerosing agent, anesthetic agent, antibiotic, enzyme, biological agent, bioabsorbable polymer, plant and animal tissue cell derivate and byproduct, vasoconstricting agent, biomaterial, conjugate, pharmaceutical agent, gene, virus, and other biochemical polymer and biological agents.

31. A formulation for injecting into a body part for treatment of a disease comprising:
a treatment substance for injection treatment of body tissue consisting of 0.05-99.9% of an active treatment substance, and 0.05-49.9% of an inactive binding substance.

32. A formulation as recited in claim 31 wherein said active treatment substance includes a chemotherapeutic agent.

33. A formulation as recited in claim 32 wherein said chemotherapeutic agent is for the purpose of tissue destruction.

34. A formulation as recited in claim 32 wherein said body tissue is a portion of a prostrate.

35. A formulation as recited in claim 34 wherein said treatment substance is for treatment of a disease selected from the group consisting of enlarged prostrate, BPH, prostatitis, prostrate cancer, prostate neurolysis, prostatic tissue and nerve destruction and other prostrate disorder.

36. A formulation comprising:
a treatment substance having a viscosity in the range of 1.0 to 10,000 cps for injection treatment of a prostate including an active treatment substance with at least one component selected from the group consisting of dehydrated ethyl alcohol (ethanol), saline, epinephrine, hypertonic saline, acetic acid, phenol, and other sclerosing agents wherein said active treatment substance is 70-99% of said treatment substance;
b) an inactive binding substance including at least one component selected from the group consisting of polymers, bioabsorbable polymers, hydroxyl propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said treatment substance.

37. A method of treating a prostate comprising:
injecting into said prostate an injectable substance having a viscosity in the range of 1.0 to 10,000 eps for injection treatment of a prostate including an active treatment substance with at least one component selected from the group consisting of dehydrated ethyl alcohol (ethanol), saline, epinephrine, hypertonic saline, acetic acid, phenol, and other sclerosing agents wherein said active treatment substance is 70-99% of said treatment substance;
b) an inactive substance including at least one component selected from the group consisting of polymers and bioabsorbable polymers, hydroxyl propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said treatment substance.

38. A method as recited in claim 37 wherein said treatment substance injected into said prostrate is in a dosage range of 0.1 to 80 cc.

39. A method as recited in claim 37 wherein said treatment substance is injected into said prostrate to treat 20 to 60% of a volume of said prostrate.

40. A formulation comprising:
a treatment substance having a viscosity in the range of 0.1 to 8000 cps for treatment of a prostrate disease selected from the group consisting of enlarged prostrate and BPH, said treatment substance including
a) at least one active treatment substance selected from the group consisting of dehydrated ethyl alcohol (ethanol), saline, epinephrine, hypertonic saline, acetic acid, phenol, and other sclerosing agents;
b) an inactive binding substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said treatment substance.

41. A method of treating a prostate comprising:
injecting into a prostate a treatment substance having a viscosity in the range of 0.1 to 8000 cps for treatment of a prostrate disease selected from the group consisting of enlarged prostrate and BPH, said treatment substance including
a) at least one active treatment substance selected from the group consisting of dehydrated ethyl alcohol (ethanol), saline, epinephrine, hypertonic saline, acetic acid, phenol, and other sclerosing agents;
b) an inactive binding substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said substance.

42. A method as recited in claim 41 wherein said treatment substance injected into said prostate is in a dosage range of 0.1 to 80 cc.

43. A method as recited in claim 41 wherein said treatment substance is injected into said prostate to treat 20 to 40% of a volume of said prostate.

44. A formulation comprising:
a) a treatment substance having a viscosity in the range of 1 to 5000 cps for treatment of prostatitis, said treatment substance including at least one active treatment substance selected from the group consisting of dehydrated ethyl alcohol (ethanol), saline, epinephrine, hypertonic saline, acetic acid, phenol, and other sclerosing agents;
b) an inactive substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said substance.

45. A method of treating a prostate comprising:
injecting into said prostate a treatment substance having a viscosity in the range of 1 to 5000 cps for treatment of prostatitis, said treatment substance including at least one active treatment substance selected from the group consisting of dehydrated ethyl alcohol (ethanol), saline, epinephrine, hypertonic saline, acetic acid, phenol, and other sclerosing agents;
b) an inactive binding substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said substance.

46. A method as recited in claim 45 wherein said treatment substance injected into said prostate is in a dosage range of 0.1 to 120 cc.

47. A method as recited in claim 45 wherein said treatment substance is injected into said prostate to treat 15 to 30% of a volume of said prostate.

48. A formulation comprising:
a treatment substance having a viscosity in the range of 500 to 10,000 cps for treatment of prostate cancer, said treatment substance including
a) at least one active treatment substance selected from the group consisting of dehydrated ethyl alcohol/ethanol, saline, epinephrine, hypertonic saline, acetic acid, phenol, and other sclerosing agents;
b) an inactive binding substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials; and
c) Epinephrine in the amount of 0 to 5.0% of said substance.

49. A method of treating a prostate comprising:
injecting into said prostate a treatment substance having a viscosity in the range of 500 to 10,000 cps for treatment of prostate cancer, said treatment substance including
a) at least one active treatment substance selected from the group consisting of dehydrated ethyl alcohol/ethanol, saline, epinephrine, hypertonic saline, acetic acid, phenol, and other sclerosing agents;
b) an inactive binding substance including at least one component selected from the group consisting of polymers and bioabsorbable polymers, hydroxy propyl cellulose (HPC), hydroxy propyl methyl cellulose (HPMC), HPEC, poly vinyl alcohol (PVA) and other rheollogy modification agents and materials; and
c) epinephrine in the amount of 0 to 5.0% of said treatment substance.

50. A method as recited in claim 49 wherein said treatment substance injected into said prostate is in a dosage of 0.1 to 150 cc.

51. A method as recited in claim 49 wherein said treatment substance is injected into said prostate to treat 30 to 80% of a volume of said prostate.

52. A formulation comprising:
a) a treatment substance having a viscosity in the range of 1 to 10,000 cps for treatment of a prostate, said treatment substance including at least one active treatment substance selected from the group consisting of the items listed in FIG. 2; and
b) an inactive binding substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials.

53. A method of treating a prostate comprising:
a) injecting into said prostate a treatment substance having a viscosity in the range of 1 to 10,000 cps for treatment of a prostate, said treatment substance including at least one active treatment substance selected from the group consisting of the items listed in FIG. 2; and
b) an inactive binding substance including at least one component selected from the group consisting of polymers, bioabsorbable polymers, hydroxy propyl cellulose (HPC), hydroxy propyl methyl cellulose (HPMC), HPEC, poly vinyl alcohol (PVA) and other rheollogy modification agents and materials.

54. A method as recited in claim 53 wherein said treatment substance injected into said prostate is in a dosage of 0.1 to 80 cc.

55. A method as recited in claim 53 wherein said treatment substance is injected into said prostate to treat 20 to 60% of a volume of said prostate.

56. A formulation comprising:
a) a treatment substance having a viscosity in the range of 1 to 10,000 cps for treatment of a prostate, said treatment substance including at least one active treatment substance selected from the group consisting of dehydrated ethyl alcohol/ethanol, saline, epinephrine, hypertonic saline, acetic acid, phenol, and other sclerosing agents;
b) an inactive binding substance including at least one component selected from the group consisting of the list of binding/gelling agents listed in FIG. 3; and
c) epinephrine in the amount of 0 to 5.0% of said substance.

57. A method of treating a prostate comprising:
a) injecting into said prostate a treatment substance having a viscosity in the range of 1 to 10,000 cps for treatment of a prostate, said treatment substance including at least one active treatment substance selected from the group consisting of dehydrated ethyl alcohol/ethanol, saline, epinephrine, hypertonic saline, acetic acid, phenol, and other sclerosing agents;
b) an inactive binding substance including at least one component selected from the group consisting of the list of binding/gelling agents listed in FIG. 3; and
c) epinephrine in the amount of 0 to 5.0% of said treatment substance.

58. A method as recited in claim 57 wherein said treatment substance injected into said prostate is in a dosage of 0.1 to 80 cc.

59. A method as recited in claim 57 wherein said treatment substance is injected into said prostate to treat 20 to 60% of a volume of said prostate.

60. A method of treating a body organ comprising:
a) inserting an injection needle apparatus interstitially into said body for injection of a treatment substance into said body organ for treatment of a disease;
b) injecting said treatment substance into said organ;
wherein said treatment substance is designed with a viscosity for control of distribution of said treatment substance, and with a composition designed for a particular concentration, composition and formulation for achieving a desired tissue effect and clinical outcome.

61. A method of treating a body part comprising:
a) inserting an injection needle into said body part, said body part selected from the group consisting of breast, uterus, fibroid, ovary, liver, kidney, lung, hemorrhoid, vocal cord, GI tract, uterine cavity, and other body parts as disclosed in FIGS. 6A and 6B; and
b) injecting said selected body part with a treatment substance as specified in FIGS. 6A and 6B.

62. A method as recited in claim 61 wherein said treatment substance is further formulated so as to be visible in real time under non-invasive imaging selected from the group consisting of CT, MRI, X-ray and ultrasound.

63. A formulation as recited in claim 36 wherein said treatment substance is further formulated so as to be visible in real time under non-invasive imaging selected from the group consisting of CT, MRI, X-ray and ultrasound.

64. A method as recited in claim 61 wherein said substance is further formulated to be electrically conductive for achieving a desired tissue effect upon application of electrical energy.

65. A method for treating a localized portion of a body part comprising:
a) inserting a needle apparatus in said body part, said apparatus including at least one hollow core needle for delivering a treatment substance into said body, said treatment substance including an inactive binding substance and an active treatment substance;
b) guiding said needle apparatus to a target tissue in said body part in need of treatment including use of an imaging device; and
c) injecting said treatment substance into said target tissue;
wherein said inserting is accomplished using instrumentation selected from the group consisting of a working channel of a biopsy guide, endoscope and laparascope.

66. A method for treating a localized portion of a body part comprising:
a) inserting a needle apparatus in said body part, said apparatus including at least one hollow core needle for delivering a treatment substance into said body, said treatment substance including an inactive binding substance and an active treatment substance;
b) guiding said needle apparatus to a target tissue in said body part in need of treatment including use of an imaging device; and
c) injecting said treatment substance into said target tissue;
wherein said guiding includes use of instrumentation selected from the group consisting of an endoscope, laparascope, ultrasound imaging instrument and grid template.

67. A method for treating a localized portion of a body part comprising:
a) inserting a needle apparatus in said body part, said apparatus including at least one hollow core needle for delivering a treatment substance into said body, said treatment substance including an inactive binding substance and an active treatment substance;
b) guiding said needle apparatus to a target tissue in said body part in need of treatment including use of an imaging device; and
c) injecting said treatment substance into said target tissue;
wherein said guiding includes use of a non-invasive method selected from the group consisting of CT, MRI, ultrasound and X-ray method.

68. A device as recited in claim 21 wherein said transurethral apparatus further includes apparatus for guiding an endoscope.

69. A device as recited in claim 68 wherein said apparatus for guiding an endoscope includes:
a) a first guide attached approximate a distal end of said tube; and
b) a second guide approximate a proximal end of said device opposite said distal end.

70. A treatment substance injection device comprising:
a) a hollow core needle for passage of said treatment substance, and wherein said needle has an echogenic tip; and
b) a transrectal ultrasound probe including a working channel for passage of said needle.

71. A device as recited in claim 70 further comprising a propulsion apparatus for propelling said treatment substance through said needle.

72. A treatment substance injection device comprising:
a) a hollow core needle for passage of said treatment substance, and wherein said needle has an echogenic tip;
b) a transrectal ultrasound probe including a needle guide attached for guiding said needle along an exterior of said probe; and
c) needle tip protection apparatus for preventing said tip from contact with body tissue during installation of said probe in a body.

73. A treatment injection device comprising:
a) a percutaneous device including
i) a hollow core needle for passage of a treatment substance, said needle having an echogenic tip; and
b) a propulsion apparatus for propelling said treatment substance through said needle.

74. A treatment device as recited in claim 73 wherein said percutaneous device is a transperinial device.

75. A device as recited in claim 73 further comprising:
a non-invasive imaging apparatus for use in guiding positioning of said needle.

76. A device as recited in claim 73 wherein said imaging apparatus is selected from the group consisting of CT, MRI, X-Ray and ultrasound apparatus.

77. A device as recited in claim 74 further comprising a non-invasive imaging apparatus for use in guiding placement of said needle.

78. A device as recited in claim 77 wherein said imaging apparatus is selected from the group consisting of CT, MRI, X-Ray and ultrasound apparatus.
